(19) United States
(12) Patent Application Publication
(10) Pub. No.: US 2026/0174839 A1
(43) Pub. Date: Jun. 25, 2026

(54) PNEUMOCOCCAL AND OTHER VACCINES POTENTIATED WITH SAPONIN ADJUVANT

(71) Applicant: THE UAB RESEARCH FOUNDATION, BIRMINGHAM, AL (US)

(72) Inventors: Pengfei WANG, Hoover, AL (US); Moon H. NAHM, Birmingham, AL (US)

(21) Appl. No.: 19/127,441

(22) PCT Filed: Nov. 6, 2023
(86) PCT No.: PCT/US2023/078771

§ 371 Date: May 6, 2025

Related U.S. Application Data
(60) Provisional application No. 63/423,164, filed on Nov. 7, 2022.

(51) Int. Cl. A61K 39/09; A61K 9/00; A61K 39/00; A61K 47/64; C12N 7/00

(52) U.S. Cl. CPC A61K 39/092; A61K 9/0019; A61K 47/646; C12N 7/00; A61K 2039/545; A61K 2039/6037

(57) ABSTRACT

In one aspect, the disclosure relates to pneumococcal vaccines comprising a semisynthetic saponin adjuvant VSA-1. In one aspect, the disclosed vaccines elicit an immune response in a subject that is measurably higher than the response in an otherwise identical subject who receives a vaccine without the semisynthetic saponin adjuvant. In another aspect, the disclosed vaccines are useful across age groups and immunization can be achieved with fewer injections than for standard pneumococcal vaccines. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/423,164, filed on Nov. 7, 2022, which is incorporated herein by reference in its entirety.

### BACKGROUND

Streptococcus pneumoniae is a leading cause of bacterial pneumonia and meningitis, accounting for an estimated 660,000 lower respiratory tract infection-related deaths and 9,600 meningitis-related deaths in adults aged >50 years of age globally each year. Mortality rates are high especially in the very young, elderly, and immunocompromised individuals. Vaccines can be an effective way to prevent infections by S. pneumoniae, including drug-resistant strains.

There are two types of clinical pneumococcal vaccines: pneumococcal polysaccharide vaccine (e.g., PPV23, composed of purified pneumococcal capsular polysaccharides of 23 serotypes of S. pneumoniae) and pneumococcal conjugate vaccine (PCV). The most commonly used PCV is PCV13, which is composed of purified capsular polysaccharide of 13 serotypes of S. pneumoniae individually conjugated to diphtheria toxin protein carrier CRM197).

PCVs are classically prepared by chemically linking capsular polysaccharide to a carrier protein. However, new approaches are being developed. In one approach, the polysaccharide and protein are linked by a ligand and receptor pair with a very high affinity (e.g., biotin and rhizavidin; MAPS technology). In another, carrier protein is prepared with a marked site for linking with a polysaccharide. This approach is used by VaxCyte. In a different approach, a polysaccharide is linked to a carrier protein by a specially designed bacterium. The conjugate vaccines prepared using bacteria are often called bio-conjugate vaccines. Furthermore, in the wake of successful PCVs, glycoconjugate vaccines are being designed for many other bacterial infections including shigella, group B streptococcus, salmonella, and E. coli.

Both types of pneumococcal vaccines have limitations. For example, PPV23 is not effective in children younger than 2 years old, the elderly, and immunocompromised individuals, and only 60-70% effective against invasive disease. The use of PCV13 substantially reduced invasive pneumococcal disease (IPD) caused by PCV13 vaccine serotypes in all age groups, but the reductions of IPD in each of the 13 vaccine serotypes of PCV13 varied among serotypes. PCV13's effectiveness against serotype 3 was not significant, and most vaccine breakthroughs in children involve serotype 3, and there are also cases involving serotypes 14 and 19A. Furthermore, PCV13 requires an inconvenient 4-dose immunization schedule for infants and young children in the United States at 2, 4, 6, and 12-15 months of age. Although other countries may require fewer doses, multiple visits to a healthcare are typically still required. In addition, immunosenescence is a noticeable issue with current pneumococcal vaccines; PCV13 is 75% effective against IPD in adults older than 65 years. It is therefore desirable to improve the efficacy of glycoconjugate vaccines.

Perhaps the most important limitation of PCV13 is that it fails to protect against non-vaccine serotypes. Consequently, there are efforts to produce a PCV with 20-30 different capsule types (B. Essink, C. Sabharwal, K. Cannon, R. Frenck, H. Lal, X. Xu, et al. Clin Infect Dis 2022 Vol. 75 Issue 3 Pages 390-398, Pivotal Phase 3 Randomized Clinical Trial of the Safety, Tolerability, and Immunogenicity of 20-Valent Pneumococcal Conjugate Vaccine in Adults Aged >/=18 Years). However, the PCVs with more serotypes than the 13 generally have lower immunogenicity than PCV13. Thus, an agent that could enhance PCV immunogenicity is highly desirable.

A viable way to potentiate humoral and cellular immune responses is to add an immunostimulating adjuvant to the vaccine. Adjuvants constitute an indispensable element of modern vaccines. They (a) enhance the ability of a vaccine to elicit strong and durable immune responses, especially in immunologically compromised individuals such as immunologically immature neonates, the aged, and immune suppressed individuals; (b) reduce antigen dose and the number of immunizations; and (c) modulate the nature of immune response. There are only a few adjuvants (i.e., alum, AS04, MF59, AS03, CpG, and AS01b) approved by the FDA for human use. PCV13 contains alum (various aluminum salts), the most used adjuvant; however, alum is a weak adjuvant and primarily enhances Th2 humoral immune responses without Th1 help. PCVs including 30 or more different serotypes are being developed. With an increasing number of serotypes, it is expected that the immunogenicity of each serotype may fall. Therefore, higher valent PCVs may especially be in need of a good adjuvant.

QS-21 is a saponin adjuvant known for its capacity of inducing both Th1 and Th2 immune responses. It was recently approved as a component of adjuvant AS01b (25, 26) used in GlaxoSmithKline's (GSK) shingles vaccine, Shingrix®, one of the most successful vaccine launches in recent years. The protection offered by QS-21 vaccines is highly durable. QS-21 vaccines are effective for broad use across age groups: Shingrix® is highly effective in older individuals (≥70 years); and the GSK's QS-21 containing malaria vaccine, MOSQUIRIX®, has been used to protect pediatric populations. However, QS-21 has its own limitations. It is a natural product isolated from the tree bark of Quillaja saponaria Molina (QS), an evergreen tree native to temperate central Chile. It has a severe supply issue; the current global supply of natural QS-21 may not be sufficient for widespread clinical use for various anti-infection vaccines. Its limited supply, along with chemical instability, dose-limiting toxicity, and laborious and low-yielding purification, hinder its wider use.

Despite advances in vaccine research, there remains an unmet medical need for more effective pneumococcal vaccines with broader serotype coverage and age group protection. An ideal adjuvant would demonstrate excellent immunostimulating properties and would be devoid of the inherent drawbacks of the FDA-approved clinical saponin adjuvant QS-21, such as limited availability, dose-limiting toxicity, chemical instability, and laborious and low-yielding purification. These needs and other needs are satisfied by the present disclosure.

### SUMMARY

In accordance with the purpose(s) of the present disclosure, as embodied and broadly described herein, the disclosure, in one aspect, relates to pneumococcal and other vaccines comprising a semisynthetic saponin adjuvant VSA-1. In one aspect, the disclosed vaccines elicit an immune response in a subject that is measurably higher than the response in an otherwise identical subject who receives a vaccine without the semisynthetic saponin adjuvant. In another aspect, the disclosed vaccines are useful across age groups and immunization can be achieved with fewer injections than for standard pneumococcal vaccines.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1G show serum antibody activity to seven serotypes, (FIG. 1A) 3, (FIG. 1B) 4, (FIG. 1C) 6B, (FIG. 1D) 9V, (FIG. 1E) 14, (FIG. 1F) 19A, and (FIG. 1G) 19F. BALB/c mice (8-10 weeks of age, six per group) were immunized via the subcutaneous route (s.c.) on days 0, 14 and 28. Serum samples were collected prior to the first immunization and at 2 weeks following the last immunization. The pooled serum samples of each group were analyzed by ELISA. The OD values of the pre-immunization serum samples of all groups (i.e., A-pre, B-pre, C-pre, and D-pre) and the post-immunization serum of group A (i.e., A-post) are in the horizontal axis. Legend in FIG. 1A is applicable to all graphs.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In pursuit of practical alternatives to QS-21, Wang et al. discovered VSA-1 adjuvant based on extensive structure-activity-relationship studies. VSA-1 is a semisynthetic saponin which can be synthesized in only one-step from naturally occurring Momordica Saponins (MS) isolated from the widely available and inexpensive seeds of Momordica cochinchinensis SPRENG (MC), aperennial vine (Synthesis of VSA-1 from MS I is depicted in Scheme 1). VSA-1 can induce a strong antigen-specific, mixed Th1/Th2 immune response mirroring QS-21 and it is much less toxic than natural QS saponins. Recently, a split virus flu vaccine showed that VSA-1 has similar/superior adjuvant activity to QS-21 in terms of stimulating humoral and cellular immune responses. Thus, it has the potential to be an effective and inexpensive alternative to QS-21 for various high-volume vaccination needs, especially for anti-infection vaccines such as, for example, pneumococcal glycoconjugate vaccines.

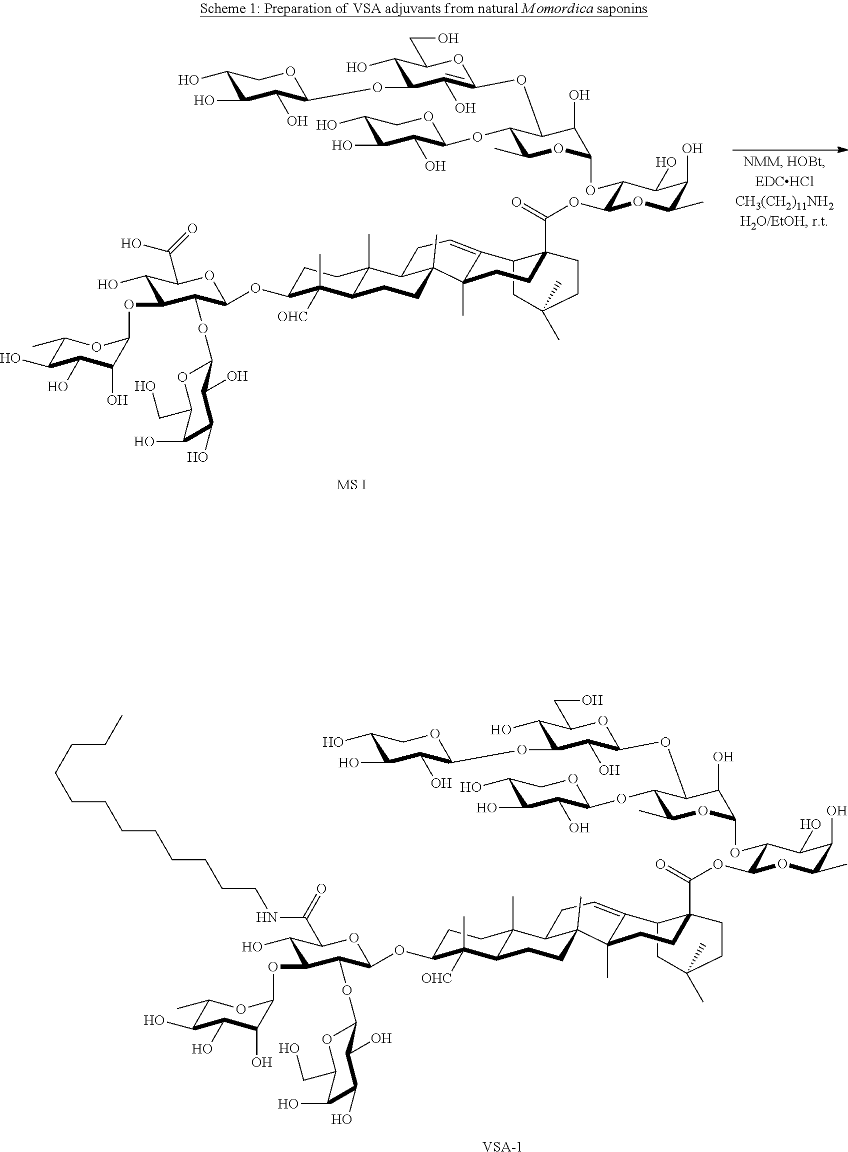

Disclosed herein are immunogenic compositions including a semisynthetic saponin adjuvant and one or more bacterial polysaccharides, wherein each of the one or more bacterial polysaccharides is linked to one or more protein carriers. In one aspect, the link can be a chemical link. In another aspect, the link uses a ligand and receptor pair having a very high affinity (e.g., biotin and rhizavidin). In still another aspect, the carrier protein can be prepared with a marked site for linking with a polysaccharide. In still another aspect, the semisynthetic saponin adjuvants such as, for example, VSA-1, are useful as adjuvants for bioconjugates.

In some aspects, the immunogenic compositions include the semisynthetic saponin adjuvant and a polysaccharide from another bacterium such as, for example, a Shigella species, a Salmonella species, Escherichia coli, a group B Streptococcus species, a Hemophilus species, or a meningococcal species. In an aspect, the semisynthetic saponin adjuvant can be VSA-1, another semisynthetic saponin derived from a Momordica saponin, a related compound, or any combination thereof. In another aspect, the semisynthetic saponin adjuvant can be present in an amount of from about 10 μg to about 1000 μg per unit dose, or at about 10, 25, 50, 75, 100, 200, 300, 400, 500, 600, 700, 800, 900, or about 1000 μg per unit dose, or a combination of any of the foregoing values, or a range encompassing any of the foregoing values.

In one aspect, the unit dose volume can be about 0.5 mL. In another aspect, at least one of the one or more bacterial polysaccharides is selected from among Streptococcus pneumoniae serotype 1, 3, 4, 5, 6A, 6B, 7F, 9V, 14, 19A, 19F, 18C, and 23F polysaccharides. In still another aspect, each one of the one or more bacterial polysaccharides is from a different Streptococcus pneumoniae serotype. In yet another aspect, the one or more protein carriers can include diphtheria toxin protein carrier CRM197.

In some aspects, the immunogenic composition includes at least one additional adjuvant such as, for example, an alum adjuvant. In another aspect, the immunogenic composition further includes at least one pharmaceutically acceptable excipient or carrier. In still another aspect, the immunogenic composition enhances both Th1 and Th2 immune responses.

Also disclosed herein is a method of eliciting an effective immune response to Streptococcus pneumoniae in a subject, the method including administering one or more unit doses of the disclosed immunogenic composition to the subject. In one aspect, the subject can be a human and administration can include intramuscular injection, subcutaneous injection, or intranasal administration. In some aspects, from at least one to at least four unit doses of the immunogenic composition can be administered to the subject. In one aspect, the two to four unit doses are administered at intervals ranging from about two weeks to about two months. In any of these aspects, the subject can be less than 2 years of age, greater than 65 years of age, or from about 2 to about 65 years of age.

In one aspect, the immune response in the subject can include at least one antigen-specific antibody titer. Further in this aspect, the at least one antigen-specific antibody titer in the subject is at least 1.5-fold higher, optionally at least 2.5-fold higher, than the same antigen-specific antibody titer in a subject to whom an otherwise identical immunogenic composition lacking the semisynthetic saponin adjuvant was administered.

In another aspect, the immune response in the subject can be measure by at least one opsonization titer. Further in this aspect, the at least one opsonization titer is at least 50% higher, or at least 100% higher, than the same opsonization titer in a subject to whom an otherwise identical immunogenic composition lacking the semisynthetic saponin adjuvant was administered.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a serotype,” “an adjuvant,” or “an antibody,” include, but are not limited to, mixtures or combinations of two or more such serotypes, adjuvants, or antibodies, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a adjuvant refers to an amount that is sufficient to achieve the desired improvement in the property modulated by the formulation component, e.g. achieving the desired enhancement of immune response in a subject to whom a vaccine containing the adjuvant is administered.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.

Aspect 1. An immunogenic composition comprising a semisynthetic saponin adjuvant and one or more polysaccharides isolated from a bacterium, wherein each one of the one or more polysaccharides is linked to one or more protein carriers.

Aspect 2. The immunogenic composition of aspect 1, wherein the bacterium comprises a Shigella species, a Salmonella species, Escherichia coli, Streptococcus pneumoniae, a group B Streptococcus species, a Hemophilus species, or a meningococcal species, or any combination thereof.

Aspect 3. The immunogenic composition of aspect 1 or 2, wherein the semisynthetic saponin adjuvant comprises VSA-1, another semisynthetic saponin derived from a Momordica saponin, or any combination thereof.

Aspect 4. The immunogenic composition of any one of aspects 1-3, wherein the semisynthetic saponin adjuvant is present in an amount of from about 10 μg to about 1000 μg per unit dose.

Aspect 5. The immunogenic composition of any one of aspects 1-4, wherein the unit dose volume comprises 0.5 mL.

Aspect 6. The immunogenic composition of any one of aspects 1-5, wherein at least one of the one or more polysaccharides is selected from among Streptococcus pneumoniae serotype 1, 3, 4, 5, 6A, 6B, 7F, 9V, 14, 19A, 19F, 18C, and 23F polysaccharides.

Aspect 7. The immunogenic composition of any one of aspects 1-6, wherein each one of the one or more polysaccharides is from a different Streptococcus pneumoniae serotype.

Aspect 8. The immunogenic composition of any one of aspects 1-7, wherein the one or more protein carriers comprise diphtheria toxin protein carrier CRM197.

Aspect 9. The immunogenic composition of any one of aspects 1-8, further comprising at least one additional adjuvant.

Aspect 10. The immunogenic composition of aspect 9, wherein the at least one additional adjuvant comprises an alum adjuvant.

Aspect 11. The immunogenic composition of any one of aspects 1-10, further comprising at least one pharmaceutically acceptable excipient or carrier.

Aspect 12. The immunogenic composition of any one of aspects 1-11, wherein the immunogenic composition enhances both Th1 and Th2 immune responses.

Aspect 13. A method of eliciting an effective immune response to Streptococcus pneumoniae in a subject, the method comprising administering one or more unit doses of the immunogenic composition of any one of aspects 1-12 to the subject.

Aspect 14. The method of aspect 13, wherein the subject is a human.

Aspect 15. The method of aspect 14, wherein the subject is less than 2 years of age.

Aspect 16. The method of aspect 14, wherein the subject is greater than 65 years of age.

Aspect 17. The method of aspect 14, wherein the subject is from 2 to 65 years of age.

Aspect 18. The method of any one of aspects 13-17, wherein administering comprises intramuscular injection. subcutaneous injection, or intranasal administration.

Aspect 19. The method of any one of aspects 13-18, comprising administering from about one to about four unit doses of the immunogenic composition to the subject.

Aspect 20. The method of aspect 19, wherein from about two to about four unit doses are administered at two week to two month intervals.

Aspect 21. The method of any one of aspects 13-20, wherein the immune response in the subject comprises at least one antigen-specific antibody titer.

Aspect 22. The method of aspect 21, wherein the at least one antigen-specific antibody titer in the subject is at least 1.5-fold higher than the same antigen-specific antibody titer in a subject to whom an otherwise identical immunogenic composition lacking the semisynthetic saponin adjuvant was administered.

Aspect 23. The method of any one of aspects 13-22, wherein the immune response in the subject can be measured by at least one opsonization titer.

Aspect 24. The method of aspect 23, wherein the at least one opsonization titer is at least 50% higher than the same opsonization titer in a subject to whom an otherwise identical immunogenic composition lacking the semisynthetic saponin adjuvant was administered.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

### Commercial Vaccines

Each human dose of PCV13 (trade name Prevnar 13 by Pfizer) is available in 0.5 mL single-dose pre-filled syringes. It contains 2.2 μg of polysaccharide from each of 12 serotypes (i.e., 1, 3, 4, 5, 6A, 7F, 9V, 14, 19A, 19F, 18C, and 23F) and 4.4 μg of polysaccharide from serotype 6B conjugated to CRM197, along with 125 μg of alum adjuvant.

### Semisynthetic Vaccine Adjuvant

Synthesis of VSA-1: A published general procedure of synthesizing MS derivatives was used. Thus, MS I (120 mg, 0.07 mmol) in ethanol (3.0 mL) and water (1.0 mL) was added dodecylamine (50.0 mg, 0.27 mmol), N-methylmorpholine (NMM) (91.0 mg, 0.90 mmol), hydroxybenzotriazole (HOBt) (83.0 mg, 0.54 mmol), and 1-ethyl-3-(3-dimethylaminopropyl) carbodiimide hydrochloride (EDC HCl) (107.0 mg, 0.54 mmol) at room temperature. The reaction mixture was stirred for 1 day, and was then filtered. The filtrate was directly purified with RP HPLC by using a Prep C18, 250×0 mm, 5-micron column, and H₂O/MeCN gradients (90%-10% H₂O over 45 minutes with a 3 mL/min flow rate). The product fraction was concentrated on a rotary evaporator at room temperature to remove MeCN, and the remaining water was then removed on a lyophilizer to provide the derivative as a white solid.

### Mice Immunizations

BALB/c mice used in this study were purchased from the Jackson Laboratory and maintained within an environmentally controlled, pathogen-free animal facility at the University of Alabama at Birmingham (UAB). Each human dose of PCV13 (trade name Prevnar 13 by Pfizer) is available in 0.5 mL single-dose pre-filled syringes. Groups of female BALB/c mice (8-10 weeks of age, six per group) were immunized via the subcutaneous route (s.c.) with saline, PCV 13 (50 μL), PCV13 (50 μL) plus QS-21 (20 μg), or PCV13 (50 μL) plus VSA-1 (100 μg) on days 0, 14 and 28. Serum samples were collected prior to the first immunization and at 2 weeks following the last immunization. Equal volumes of the six sera in each group were pooled together to create serum pools for each group. The serum was obtained after centrifugation and stored at −20° C. until assayed. All studies were performed according to National Institutes of Health guidelines, and protocols were approved by the UAB Institutional Animal Care and Use Committee.

### ELISA

An assay described for human serum was adapted for mouse serum as described below. Briefly, each well of a 96-well microtiter plate was coated with 100 μL of PBS with a capsular PS at a pre-determined concentration. Capsular PS of the seven serotypes (3, 4, 6B, 9V, 14, 19A, 19F, and 22F) were from ATCC. Plates were incubated at 37° C. for 5 h in a humidified chamber, except type 3 PS which was coated at room temperature for 2 hours. The coated plates were washed with Washing Buffer (TBS-0.1% Brij-35 solution) and blocked with PBS containing 0.5% BSA, 0.05% Tween 20 and 0.02% NaN₃. To the PS-coated microtiter plates, was loaded 50 μL of the serum pool diluted as below. The serum pools were made by mixing equal volume of individual mouse serum in each group. The resulting serum pools were initially diluted 1:50 and then 3-fold serially diluted in Antibody Buffer (PBS with 0.1% BSA, 0.05% Tween-20 and 0.02% NaN₃) with 5 μg/ml of teichoic acid (the Statens Serum Institut in Denmark) and 5 μg/mL 22F capsule (ATCC). The plates were incubated overnight at room temperature in a humidified box. After washing five times, 100 μL of diluted alkaline phosphatase-conjugated goat mouse immunoglobulin (Southern Biotech, Birmingham, AL) in Antibody Buffer was added to each well. After another 1-h incubation, the plates were washed five times, and 100 μL of the substrate solution containing p-nitrophenyl phosphate (Sigma) was added to each well. After a 1-h incubation at room temperature, the optical density was measured at 405 nm and at 690 nm.

### Opsonophagocytosis Assay

Opsonophagocytosis assay for serotypes 3, 4, 6B, 9V, 14, 19A, and 19F was performed using a 4-fold multiplexed opsonization assay described in the literature. Briefly, 10 μL of bacterial suspension (˜2×10⁵ CFU/mL of each serotype) and 20 μL of serially diluted antiserum were incubated in a microtiter plate for 30 min at RT with shaking. Then 10 μL of 3- to 4-week-old rabbit serum as the complement source (PelFreeze Biologicals, Rogers, AK) and 40 μL of differentiated HL60 cells (4×10⁵ cells) were added to each well and the plates were incubated at 37° C. in 5% CO² with shaking for 45 min. An aliquot of the final reaction mixture (10 μL) was spotted onto four different THY agar plates, and overlay agar containing one of the four antibiotics (optochin, spectinomycin, streptomycin, or trimethoprim) was applied to each THY agar plate. After an overnight incubation at 37° C., the number of bacterial colonies was enumerated. Opsonic indices were determined as the interpolated serum dilution that kills 50% of bacteria.

### Example 2: Results and Discussion

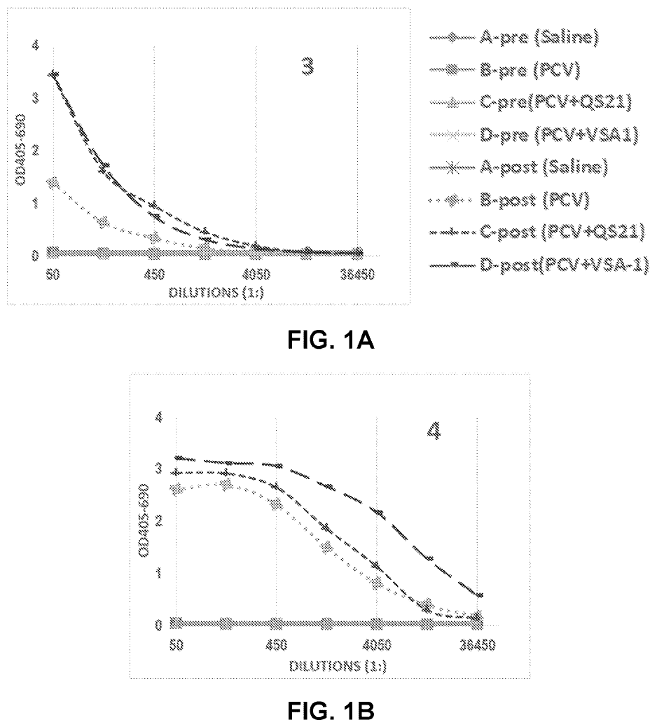

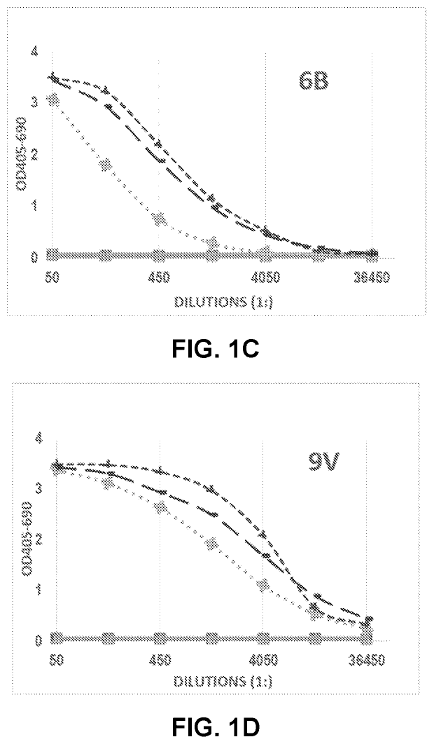

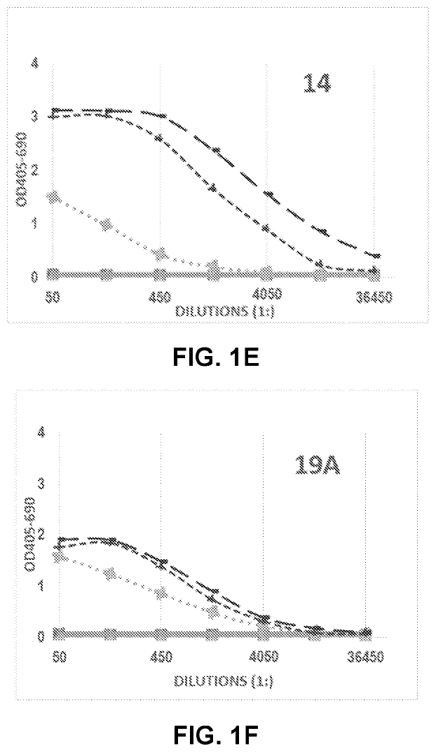

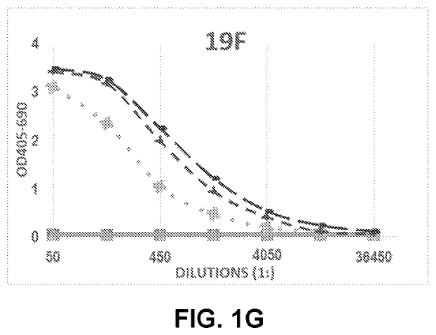

Herein are reported results of comparing VSA-1 and QS-21 in enhancing the immune responses induced by the clinical glycoconjugate pneumococcal vaccine PCV13. Each human dose of PCV13 (trade name Prevnar 13 by Pfizer) is available in 0.5 mL single-dose pre-filled syringes. It contains 2.2 μg of polysaccharide from each of 12 serotypes (i.e., 1, 3, 4, 5, 6A, 7F, 9V, 14, 19A, 19F, 18C, and 23F) and 4.4 μg of polysaccharide from serotype 6B conjugated to CRM197, along with 125 μg of alum adjuvant. Thus, groups of female BALB/c mice (8-10 weeks of age, six per group) were immunized via the subcutaneous route (s.c.) with saline (group A, negative control), PCV 13 (group B), PCV13 plus QS-21 (20 μg) (group C), or PCV13 plus VSA-1 (100 μg) (group D) on days 0, 14 and 28. One tenth of one human dose of PCV13 was used for each mouse dose. Serum samples were collected prior to the first immunization and at 2 weeks following the last immunization. Equal volumes of the six sera in each group were pooled together to create serum pools for each group. ELISA was used to assess the antibody activity toward seven PCV13 serotypes, i.e., 3, 4, 6B, 9V, 14, 19A, and 19F (FIGS. 1A-1G).

The serum samples collected from all the four groups prior to the first immunization (Pre-Immune) and the serum of group A (saline control) post the last immunization (Post-A) showed no antigen-specific antibody titers (Table 1, FIGS. 1A-1G). PCV13 induced significant antibody responses to most of the seven tested serotypes (Post-B), and inclusion of a saponin adjuvant to PCV13 enhanced antigen-specific antibody responses to all eight serotypes compared with the PCV13 control group. When comparing the dilutions that gave OD of 4×background, VSA-1 (Post-D) was superior to QS-21 (Post-C) for serotypes 4, 6B, 9V, 14, 19A, and 19F (Table 1).

TABLE 1
ELISA Titer to Different Vaccine Serotypes^{a}

| | Serotypes | | | | | | |
|---|---|---|---|---|---|---|---|
| Sera | 3 | 4 | 6B | 9V | 14 | 19A | 19F |
| Pre-immune | <50 | <50 | <50 | <50 | <50 | <50 | <50 |
| Post-A (Saline) | <50 | <50 | <50 | <50 | <50 | <50 | <50 |
| Post-B (PCV Only) | 810 | 24300 | 1800 | 36450 | 1035 | 3375 | 3375 |
| Post-C (+QS21) | 3375 | 18225 | 8910 | 72900 | 12150 | 6075 | 8100 |
| Post-D (+VSA1) | 2160 | >109350 | 11340 | 91125 | 91125 | 80100 | 12150 |

^{a}Dilution that gives OD of 4× background.

The ELISA results provided evidence of antibody responses to the different vaccine formulations; however, these data do not indicate the ability of the antibodies to opsonize and kill bacteria, thus do not provide direct evidence of immune protection. Vaccine-induced immune protection against encapsulated S. pneumoniae is primarily mediated by opsonic antibodies that bind capsular polysaccharide antigens (CPSs). Opsonophagocytosis assay (OPA) is an important tool to evaluate the capacity of sera to kill the bacteria. OPA for serotypes 3, 4, 6B, 9V, 14, 19A, and 19F was performed using a 4-fold multiplexed opsonization assay. Opsonic titers are defined as the reciprocal of the interpolated serum dilution that kills 50% of the bacteria. The OPA data show that inclusion of a saponin adjuvant in PCV13 enhance opsonic titers for all serotypes (Table 2). With QS-21, PCV13-induced opsonic titers increased in the range of 1.9-4.9 fold for serotypes 3, 4, 6B, 9V, 19A, and 19F, and a 14.9-fold increase for serotype 14. VSA-1 improved opsonic titers against serotype 14 even more, with an 18.2-fold increase, and a 2.1-4.1 fold increase for other serotypes except for serotypes 9V (×0.8), even though it increased IgG response against 9V by 2.5 fold compared with PCV13 alone (Table 1). Comparison between VSA-1 and QS-21 shows that VSA-1 is superior to QS-21 in enhancing opsonic titers for serotypes 4, 6B, 14, and 19A, by 1.8, 2.0, 1.2, and 1.1 fold, respectively. QS-21 is superior to VSA-1 for serotypes 3, 9V, and 19F, by 1.3, 2.4, and 1.2 fold, respectively.

TABLE 2
OPI Titer to Different Vaccine Serotypes^{a}

| | Serotypes | | | | | | |
|---|---|---|---|---|---|---|---|
| Sera | 3 | 4 | 6B | 9V | 14 | 19A | 19F |
| Pre-immune | <20 | <20 | <20 | <20 | 110 | <20 | <20 |
| Post-A (Saline) | <20 | <20 | <20 | <20 | 83 | <20 | <20 |
| Post-B (PCV Only) | 673 | 7124 | 2511 | 4613 | 2398 | 1469 | 926 |
| Post-C (+QS21) | 3288 | 11298 | 5226 | 9235 | 35835 | 2779 | 2836 |
| Post-D (+VSA1) | 2514 | 19825 | 10296 | 3775 | >43740^{b} | 3054 | 2343 |

^{a}The opsonization titer was defined as the highest dilution of the serum that kills 50% of the target bacteria
^{b}Post-D has high serotype 14 killing titer that exceeded the upper limit of the assay.

The ELISA and OPA data suggest that adding saponin adjuvant VSA-1 to PCV13 or other PCVs is a viable way to boost antibody responses and increase opsonic antibodies induced by PCV13 or other PCVs. Both VSA-1 and QS-21 boosted IgG and OPA titers against the tested serotypes, including serotypes 3, 14, and 19A that are involved in most PCV13 breakthroughs, especially serotype 3. VSA-1 and QS-21 are known for stimulating antigen-specific humoral and cellular immunity. They can potentially enhance the serotype-specific immune memory and help to reduce number of immunizations of PCV13 while maintaining a high level of protection. The adjuvants' capability in stimulating cellular immune responses can also help to overcome immunosenescence and improve efficacy of glycoconjugate pneumococcal vaccine in the elderly, which is important given that PCV13 is 75% effective against PID in adults older than 65 years. Since VSA-1 is much more accessible and of lower toxicity than QS-21, it can be a practical saponin immunostimulant to be included in a new glycoconjugate pneumococcal vaccine formulation.

### Example 3: VSA-1 to Enhance Immunogenicity of PCV13 and IgG2a Antibody Response

### Background

Streptococcus pneumoniae is an important human pathogen and pneumococcal conjugate vaccines (PCVs) have been highly effective against the serotypes included in the vaccine. However, infections by the non-vaccine serotypes have become more common and PCVs with twenty or more serotypes are being developed. Since inclusion of more serotypes has greatly reduced the immunogenicity of PCVs, a novel adjuvant is desirable. We have therefore examined a new adjuvant (VSA-1) for enhancing immunogenicity of PCV13 and IgG2a antibody response. VSA-1 is a semisynthetic saponin adjuvant prepared from naturally occurring and abundantly available Momordica saponin.

### Methods

Four groups of female BALB/c mice (6 per group) were immunized with normal saline, one tenth human dose of PCV13, PCV13+VSA-1, and PCV13-QS21 on days 0, 14 and 14. QS21 is another saponin adjuvant. Blood samples were obtained on day 42. IgG subclass of pneumococcal antibodies in the pooled serum samples of each group were determined with pneumococcal antibody ELISA for seven serotypes (3, 4, 6B, 9V, 14, 19A and 19F) using enzyme-conjugated antibodies against mouse IgG1, IgG2a or IgG3.

### Results

Both VSA-1 and QS-21 boosted IgG1, IgG2a and IgG3 antibodies titers against seven selected serotypes (Table 3).

TABLE 3
Fold Increase in ELISA Titer of Each IgG Subclass over Saline Group^{a}

| | | Serotype | | | | | | | Average |
|---|---|---|---|---|---|---|---|---|---|
| | Group | 3 | 4 | 6B | 9V | 14 | 19A | 19F | (n = 7) |
| IgG1 | A (Saline) | 1 | 1 | 1 | 1 | 1 | 1 | 1 | 1 |
| | B (PCV only) | 27 | 737 | 39 | 1056 | 42 | 102 | 119 | 303 |
| | C (PCV + QS21) | 119 | 1370 | 300 | 2187 | 793 | 173 | 175 | 731 |
| | D (PCV + VSA1) | 112 | 2187 | 222 | 2187 | 1741 | 219 | 236 | 986 |
| IgG2a | A (Saline) | 1 | 1 | 1 | 1 | 1 | 1 | 1 | 1 |
| | B (PCV only) | 2 | 3 | 1 | 17 | 1 | 2 | 1 | 4 |
| | C (PCV + QS21) | 10 | 71 | 8 | 678 | 17 | 14 | 15 | 116 |
| | D (PCV + VSA1) | 5 | 96 | 1 | 618 | 216 | 23 | 49 | 144 |
| IgG3 | A (Saline) | 1 | 1 | 1 | 1 | 1 | 1 | 1 | 1 |
| | B (PCV only) | 7 | 30 | 2 | 29 | 22 | 1 | 14 | 15 |
| | C (PCV + QS21) | 18 | 165 | 8 | 402 | 360 | 49 | 246 | 178 |
| | D (PCV + VSA1) | 19 | 182 | 6 | 301 | 2187 | 44 | 15 | 393 |

^{a}The titer is defined as the serum dilution that produced 6× above the background signal.

VSA-1 is as effective as QS21 in enhancing antibody response to PCV13 and significantly increasing IgG2a antibody response. While additional studies are required, VSA-1, which is much more accessible and of lower toxicity than QS-21, appears to stimulate Th1 as QS-21 does.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Andrews N J, et al. Serotype-specific effectiveness and correlates of protection for the 13-valent pneumococcal conjugate vaccine: a postlicensure indirect cohort study. Lancet Infect Dis. 2014; 14:839-46.
2. Backhaus E, et al. Epidemiology of invasive pneumococcal infections: manifestations, incidence, and case fatality rate correlated to age, gender, and risk factors. BMC Infect Dis 2016; 16:367.
3. Bonam S R, et al. An overview of novel adjuvants designed for improving vaccine efficacy. Trends Pharmacol Sci. 2017; 38(9):771-93.
4. Burton R L et al. Development and validation of a fourfold multiplexed opsonization assay (MOPA4) for pneumococcal antibodies. Clin Vaccine Immunology. 2006; 13(9):1004-9.
5. Burton R L et al. Development of a fourfold multiplexed opsonophagocytosis assay for pneumococcal antibodies against additional serotypes and discovery of serological subtypes in Streptococcus pneumoniae serotype 20. Clin Vaccine Immunol. 2012; 19(6):835-41.
6. Cohen R, et al. Invasive disease potential of pneumococcal serotypes in children after PCV13 implementation. Clin Infect Dis. 2021; 72(8):1453-6.
7. Corcoran M, et al. The persistence of serotype 19A-Despite the introduction of PCV13 vaccine. 10th International symposium on pneumococci & pneumococcal disease; Glasgow, U K 2016.
8. Cunningham A L, et al. Efficacy of the Herpes Zoster Subunit Vaccine in Adults 70 Years of Age or Older. The New England Journal of Medicine. 2016; 375(11):1019-32.
9. Di Pasquale A, et al. Vaccine adjuvants: from 1920 to 2015 and beyond. Vaccines (Basel). 2015; 3(2):320-43.
10. Didierlaurent A M, et al. Adjuvant system AS01: helping to overcome the challenges of modern vaccines. Expert Rev Vaccines. 2017; 16(1):55-63.
11. Dominguez Â, et al. Effectiveness of the 13-valent pneumococcal conjugate vaccine in preventing invasive pneumococcal disease in children aged 7-59 months. A matched case-control study. PLOS One. 2017; 12(8):e0183191.
12. Dransfield M T, et al. Long-term comparative immunogenicity of protein conjugate and free polysaccharide pneumococcal vaccines in chronic obstructive pulmonary disease. Clin Infect Dis. 2012; 55(5):e35-44.
13. Garcon N, et al. Recent clinical experience with vaccines using MPL- and QS-21-containing adjuvant systems. Expert Rev Vaccines. 2011; 10(4):471-86.
14. Garçon N, et al. Vaccine Adjuvants. In: Garçon N et al, editors. Understanding Modern Vaccines Perspectives in Vaccinology Amsterdam: Elsevier; 2011. p. 89-113.
15. Giudice G D, et al. Correlates of adjuvanticity: A review on adjuvants in licensed vaccines. Semin Immunol. 2018; 2018:14-21.
16. Gruber W C, et al. Development and clinical evaluation of Prevnar 13, a 13-valent pneumocococcal CRM197 conjugate vaccine. Ann N Y Acad Sci. 2012; 1263:15-26.
17. Horacio A N, et al. Serotype 3 remains the leading cause of invasive pneumococcal disease in adults in portugal (2012-2014) despite continued reductions in other 13-valent conjugate vaccine serotypes. Front Microbiol. 2016; 7.
18. Jackson L A, et al. Randomized clinical trial of a single versus a double dose of 13-valent pneumococcal conjugate vaccine in adults 55 through 74 years of age previously vaccinated with 23-valent pneumococcal polysaccharide vaccine. Vaccine. 2018; 36(5):606-14.
19. James S F, et al. Shingrix: the new adjuvanted recombinant herpes zoster vaccine. Annals of Pharmacotherapy. 2018; 52(7):673-80.
20. Kim Y K, et al. Indirect effects of pneumococcal conjugate vaccines in national immunization for children on adult pneumococcal disease. Infect Chemother. programs 2016; 48(4):257-66.
21. Lacaille-Dubois M A. Updated insights into the mechanism of action and clinical profile of the immunoadjuvant QS-21: A review. Phytomedicine. 2019; 60:152905.
22. LaFon D C et al. Measuring quantity and function of pneumococcal antibodies in immunoglobulin products. Transfusion. 2018; 58 Suppl 3:3114-20.
23. LaFon D C, et al. Measuring immune responses to pneumococcal vaccines. J Immunol Methods. 2018; 461:37-43.
24. Lee L H, et al. Towards new broader spectrum pneumococcal vaccines: The future of pneumococcal disease prevention. Vaccines (Basel). 2014; 2(1):112-28.
25. Leroux-Roels I, et al. Adjuvant system AS02V enhances humoral and cellular immune responses to pneumococcal protein PhtD vaccine in healthy young and older adults: randomised, controlled trials. Vaccine. 2015; 33(4):577-84.
26. Ludwig G, et al, Catalan Study Group of Invasive Pneumococcal D. Serotype and clonal distribution dynamics of invasive pneumococcal strains after PCV13 introduction (2011-2016): Surveillance data from 23 sites in Catalonia, Spain. PLOS One. 2020; 15(2):e0228612.
27. Martin R S, et al. Industrial uses and sustainable supply of Quillaja Saponaria (Rosaceae) Saponins. Economic Botany. 1999; 53(3):302-11.
28. Masomian M, et al. Development of next generation Streptococcus pneumoniae vaccines conferring broad protection. Vaccines (Basel). 2020; 8(1).
29. Mokaddas E, et al. The 13-valent pneumococcal conjugate vaccine (PCV13) does not appear to provide much protection on combined invasive disease due to the six PCV13 non-PCV7 serotypes 1, 3, 5, 6A, 7F, and 19A in Kuwait during 2010-2019. Hum Vaccin Immunother. 2021; 17(11):4661-6.
30. Morefield G L. A rational, systematic approach for the development of vaccine formulations. AAPS J. 2011; 13(2):191-200.
31. Mungall B A, et al. A systematic review of invasive pneumococcal disease vaccine failures and breakthrough with higher-valency pneumococcal conjugate vaccines in children. Expert Rev Vaccines. 2022; 21(2):201-14.
32. O'Hagan D T, et al. Towards an evidence based approach for the development of adjuvanted vaccines. Current Opinion in Immunology. 2017; 47:93-102.
33. Ragupathi G, et al. Natural and synthetic saponin adjuvant QS-21 for vaccines against cancer Expert Rev Vaccines. 2011; 10:463-70.
34. Shi S, et al. Vaccine adjuvants: Understanding the structure and mechanism of adjuvanticity. Vaccine. 2019; 37:3167-78.
35. Silva-Costa C, et al. Continued Vaccine Breakthrough Cases of Serotype 3 Complicated Pneumonia in Vaccinated Children, Portugal (2016-2019). Microbiology Spectrum. 2022. doi: 10.1128/spectrum.01077-22.
36. Song J Y et al. Pneumococcal vaccine and opsonic pneumococcal antibody. J Infect Chemother. 2013; 19:412-25.
37. Song J Y et al. Pneumococcal vaccine and opsonic pneumococcal antibody. J Infect Chemother. 2013; 19(3):412-25.
38. Sterrett S, et al. Peripheral CD4 T follicular cells induced by a conjugated pneumococcal vaccine correlate with enhanced opsonophagocytic antibody responses in younger individuals. Vaccine. 2020; 38:1778-86.
39. Theilacker C, et al. PCV13 Vaccination of Adults against Pneumococcal Disease: What We Have Learned from the Community-Acquired Pneumonia Immunization Trial in Adults (CAPITA). Microorganisms. 2022; 10(1).
40. Wang P, et al. Synthesis and evaluation of a QS-17/18-based vaccine adjuvant. J Med Chem. 2019; 62:1669-76.
41. Wang P, et al. Synthesis and evaluation of QS-21-based immunoadjuvants with a terminal-functionalized side chain incorporated in the west wing trisaccharide. J Org Chem. 2016; 81:9560-6.
42. Wang P, et al. Synthesis and evaluation of QS-7-based vaccine adjuvants. ACS Infectious Diseases. 2019; 5:974-81.
43. Wang P, et al. Synthesis of QS-21-based immunoadjuvants. J Org Chem. 2013; 78:11525-34.
44. Wang P, et al. Vaccine adjuvants derivatized from Momordica saponins I and II. J Med Chem. 2019; 62:9976-82.

1. An immunogenic composition comprising a semisynthetic saponin adjuvant and one or more polysaccharides isolated from a bacterium, wherein each one of the one or more polysaccharides is linked to one or more protein carriers; wherein the semisynthetic saponin adjuvant comprises VSA-1, another semisynthetic saponin derived from a Momordica saponin, or any combination thereof.

2. The immunogenic composition of claim 1, wherein the bacterium comprises a Shigella species, a Salmonella species, Escherichia coli, Streptococcus pneumoniae, a group B Streptococcus species, a Hemophilus species, or a meningococcal species, or any combination thereof.

3. (canceled)

4. The immunogenic composition of claim 1, wherein the semisynthetic saponin adjuvant is present in an amount of from about 10 μg to about 1000 μg per unit dose.

5. The immunogenic composition of claim 1, wherein a unit dose volume comprises 0.5 mL.

6. The immunogenic composition of claim 2, wherein at least one of the one or more polysaccharides is selected from among Streptococcus pneumoniae serotype 1, 3, 4, 5, 6A, 6B, 7F, 9V, 14, 19A, 19F, 18C, and 23F polysaccharides.

7. The immunogenic composition of claim 2, wherein each one of the one or more polysaccharides is from a different Streptococcus pneumoniae serotype.

8. The immunogenic composition of claim 1, wherein the one or more protein carriers comprise diphtheria toxin protein carrier CRM197.

9. The immunogenic composition of claim 1 further comprising at least one additional adjuvant, wherein the at least one additional adjuvant comprises an alum adjuvant.

10. (canceled)

11. (canceled)

12. The immunogenic composition of claim 1, wherein the immunogenic composition enhances both Th1 and Th2 immune responses.

13. A method of eliciting an effective immune response to Streptococcus pneumoniae in a subject, the method comprising administering one or more unit doses of the immunogenic composition of claim 1 to the subject.

14. The method of claim 13, wherein the subject is a human.

15. The method of claim 14, wherein the subject is less than 2 years of age.

16. The method of claim 14, wherein the subject is greater than 65 years of age.

17. The method of claim 14, wherein the subject is from 2 to 65 years of age.

18. The method of claim 13, wherein administering comprises intramuscular injection. subcutaneous injection, or intranasal administration.

19. The method of claim 13, comprising administering from about one to about four unit doses of the immunogenic composition to the subject.

20. The method of claim 19, wherein from about two to about four unit doses are administered at two week to two month intervals.

21. The method of claim 13, wherein the immune response in the subject comprises at least one antigen-specific antibody titer.

22. The method of claim 21, wherein the at least one antigen-specific antibody titer in the subject is at least 1.5-fold higher than the same antigen-specific antibody titer in a subject to whom an otherwise identical immunogenic composition lacking the semisynthetic saponin adjuvant was administered.

23. The method of claim 13, wherein the immune response in the subject can be measured by at least one opsonization titer, wherein the at least one opsonization titer is at least 50% higher than the same opsonization titer in a subject to whom an otherwise identical immunogenic composition lacking the semisynthetic saponin adjuvant was administered.

24. (canceled)
